(19) United States
(12) Patent Application Publication
(10) Pub. No.: US 2026/0002923 A1
(43) Pub. Date: Jan. 1, 2026

(54) TESTING DEVICE AND METHOD FOR MEASURING OIL, GAS AND WATER CONTENTS IN ROCK

(71) Applicants: CHINA PETROLEUM & CHEMICAL CORPORATION, Beijing (CN); SINOPEC EXPLORATION & PRODUCTION RESEARCH INSTITUTE, Beijing (CN)

(72) Inventors: Qigui JIANG, Beijing (CN); Peng LIU, Beijing (CN); Lingjie YU, Beijing (CN); Changbo ZHAI, Beijing (CN); Cheng TAO, Beijing (CN); Longfei LU, Beijing (CN); Yane CHEN, Beijing (CN)

(21) Appl. No.: 18/992,208

(22) PCT Filed: Feb. 6, 2023
(86) PCT No.: PCT/CN2023/074529

§ 371 Date: Jan. 8, 2025

(30) Foreign Application Priority Data
Jul. 8, 2022 (CN) 202210806887.8

(51) Int. Cl. G01N 33/24

(52) U.S. Cl. CPC G01N 33/241; G01N 33/246

(57) ABSTRACT

A testing device for measuring oil, gas and water contents in rock has a heating desorption unit that has a sample chamber for placing a rock sample to be detected, a crushing mechanism arranged within the sample chamber, and a heating mechanism. The heating desorption unit crushes and heats the rock sample in a sealed manner to achieve gas analysis and desorption analysis of oil and water contained therein, thus obtaining components to be detected. The device further has a cold trap collection unit in communication with the sample chamber, a light hydrocarbon capture unit in communication with the cold trap collection unit, a gas measuring unit in communication with the light hydrocarbon capture unit for measuring and calculating a gas volume; and a central control unit for controlling the testing device.

### CROSS REFERENCE OF RELATED APPLICATIONS

The present application claims the priority of Chinese patent application No. 202210806887.8 entitled “Device for detecting contents of oil, gas and water in rock and application thereof” and filed on Jul. 8, 2022, the entire content of which is incorporated herein by reference.

### TECHNICAL FIELD

The present invention relates to the technical field of oil/gas exploration and development, and specifically to a testing device and method for measuring oil, gas and water contents in rock.

### TECHNICAL BACKGROUND

Rock is a carrier with occurrence of oil, gas and water. Accurate information on oil, gas and water contents in rock and components thereof is of great significance in the field of oil/gas exploration and development as well as environmental protection, which can provide direct evidence for the evaluation of oil/gas potential and mobility in rocks, thus guiding the selection of stratum segment in oil/gas field and the formulation of development plans thereof. In the field of environmental protection, the contents and components of oil, gas and water in soil can serve as a scientific basis for decisions on improvement of the environment.

The analysis methods for oil content in rock can be mainly divided into two categories, i.e., solvent extraction method and pyrolysis method. The solvent extraction method further includes powder sample extraction and plug sample (block sample) extraction, and the pyrolysis method also includes powder sample pyrolysis and gas thermal elution of block sample. Powder sample is most widely used in both solvent extraction method and pyrolysis method, for which national and industry standards have also been released, for example, the national standard CN SY/T 5118 “Determination of bitumen from rocks by chloroform extraction”, and CN GB/T 18602 “Rock pyrolysis analysis”. The device mainly used in the solvent extraction method is the Soxhlet extractor, while the device mainly used in the rock pyrolysis method is the pyrolyzer, such as ROCK-EVAL rock pyrolyzer (France), HAWK rock pyrolyzer (U.S.), and some other rock evaluation instruments (China). However, few oil potential experiments have been carried out on block rock samples. The existing washing oil experiments are mainly directed to analyze the porosity of block rock samples, and oil content in the rock can be directly obtained from quantitative results of washing oil. For example, CN104668233A discloses a rock core washing oil instrument, wherein the solvent dissolves and elutes soluble organic matters in rock. The eluent can be recycled repeatedly after distilling and cooling the solvent, and oil content in the rock can be obtained by weighing after the solvent volatilizes.

With the increasing demand for shale gas exploration and development, the analysis methods of gas content in rock have developed rapidly, encouraging the emergence of various analysis technologies and devices for shale gas content. For example, CN10468076A discloses a testing device and method for shale gas content, wherein core samples are pulverized and degassed by a combined crushing tool in a sealed chamber, so as to obtain the gas content in rock quickly. As another example, CN104155206A discloses a device for measuring gas content in rock and a method for measuring gas content in rock using the same, wherein the gas in the rock is desorbed through heating, and the gas volume is automatically measured by drainage gas recovery. The molecular sieve cold trapping technology and the thermal desorption and capture technology are frequently used for the analysis of gas in soil.

There is a variety of methods for analyzing water content in substances. The analysis methods for water content in rock or soil generally fall into two categories, i.e., distillation method and electric measurement method. The distillation method has a wide range of applications. For example, CN2061146866U discloses a device for measuring water content in soil, which obtains water content by combining multiple drying processes and an electronic scale. CN207586066U discloses a device for measuring water content in rock core, wherein the core is heated with a dry distiller to distill gas and liquid, through which liquid is collected via a cooling system. Then oil and water are separated from the liquid, in order to measure the water content. The electric measurement method is also widely applied in the determination of water content in soil, but rarely applied in the determination of water content in rock. A typical example is CN109459333A, which discloses a portable soil moisture and unit weight measuring device and a soil moisture and unit weight measuring method, which is based on the principle that electromagnetic wave has different propagation velocities in different media. A UWB sensor is placed on both sides of an object to be measured, and releases electromagnetic wave which passes through the soil. There is a linear relationship between the water content in soil and the data measured by the UWB sensor. Moreover, CN211856430U discloses a soil moisture content tester, wherein two measuring needles are inserted into the soil to be measured, in order to obtain the soil moisture content based on internal conductivity.

The foregoing indicates that in spite of the various analysis devices and methods for oil, gas and water contents in rock, they are independent of each other. Therefore, multiple separate experimental devices are required for respective measurements, in order to obtain oil, gas and water contents in the rock sample. Due to the dissipation property of gas, light hydrocarbon and moisture, as well as the heterogeneity of samples, the analysis results obtained by combining multiple methods together may not match with each other, thus leading to uncertain geological interpretation.

### SUMMARY OF THE INVENTION

In view of the above technical problems, the present invention aims to propose a testing device and method for measuring oil, gas and water contents in rock.

According to a first aspect of the present invention, a testing device for measuring oil, gas and water contents in rock is proposed, which comprises a beating desorption unit, comprising a sample chamber for placing a rock sample to be detected, a crushing mechanism arranged within the sample chamber, and a heating mechanism, the heating desorption unit being configured to crush and heat the rock sample in a sealed manner, in order to achieve gas analysis and desorption analysis of oil and water contained therein, thus obtaining components to be detected; a cold trap collection unit in communication with the sample chamber, for capturing and separating oil and water from the components to be detected, in order to obtain preliminarily separated components to be detected; a light hydrocarbon capture unit in communication with the cold trap collection unit, for capturing and separating light hydrocarbon from the preliminarily separated components to be detected, in order to obtain secondarily separated components to be detected; a gas measuring unit in communication with the light hydrocarbon capture unit, for measuring and calculating a gas volume; and a central control unit, for controlling the testing device.

In one embodiment, the gas measuring unit includes a liquid storage tank, at least one measuring tube in communication with the liquid storage tank, a magnetostrictive assembly arranged inside the measuring tube, and a magneto detection assembly, wherein the magnetostrictive assembly is configured to generate magnetostrictive effect and twist when the gas is introduced into the measuring tube and a liquid level in the measuring tube changes, and the magneto detection assembly is configured to generate an initial pulse, and a corresponding return pulse according to a twist of the magnetostrictive assembly, whereby a liquid displacement and further the gas volume are calculated.

In one embodiment, the magnetostrictive assembly comprises a detecting rod vertically arranged inside the measuring tube; a magnetostrictive wire arranged inside the detecting rod, wherein a rotating magnetic field is generated when the initial pulse generated by the magneto detection assembly moves along the magnetostrictive wire; and a movable magnetic floating ring, which is sleeved outside the detecting rod and configured to move according to changes in the liquid level, for generating the magnetostrictive effect when encountering the rotating magnetic field.

In one embodiment, a pressure sensor and a temperature sensor are further arranged within the measuring tube.

In one embodiment, the sample chamber includes a tank body and a crushing tank cover connected to each other in a sealed manner through a first sealing packing.

In one embodiment, the crushing mechanism includes a primary crushing unit connected to the crushing tank cover and a secondary crushing unit arranged inside the tank body and at a bottom portion thereof, wherein the primary crushing unit is configured to impact on and crush the rock sample, in order to obtain crushed rock samples, and the secondary crushing unit is configured to attrite and grind the crushed rock samples.

In one embodiment, the primary crushing unit includes a push rod, a counterweight connected to a lower end of the push rod, an energy storage element arranged on the push rod, and a driving assembly, an upper end of the push rod passing through the crushing tank cover and extending outward to connect to the driving assembly, wherein the counterweight is lifted up through the driving assembly, enabling the energy storage element to store energy, and then released to impact on and crush the rock sample.

In one embodiment, a dynamic seal is formed between the push rod and the crushing tank cover through a dynamic seal assembly, wherein the dynamic seal assembly includes a seal fixing seat and a pneumatic sealing ring installed thereon, the seal fixing seat passing through a central area of the crushing tank cover, so that the dynamic seal is formed between the push rod and the crushing tank cover through the pneumatic sealing ring.

In one embodiment, the secondary crushing unit includes a vibrating plate, grinding balls arranged on the vibrating plate, and an electromagnetic vibrator for driving the vibrating plate, wherein the vibrating plate is configured to work in cooperation with the grinding balls to attrite and grind the crushed rock samples.

In one embodiment, a heat insulation plate is further fixed inside the tank body and connected to a lower end of the crushing tank cover through a fixing rod, wherein the push rod passes through the heat insulation plate, with a dynamic seal formed therebetween through a second sealing packing.

In one embodiment, the cold trap collection unit comprises a cold trap capture mechanism, for capturing the oil and water in the components to be detected; and a low-temperature cold trap, for providing a low-temperature environment for the cold trap capture mechanism, the cold trap capture mechanism being arranged within the low-temperature cold trap.

In one embodiment, the cold trap capture mechanism includes a first capture tube, a sealing cover for sealing the first capture tube, and an air inlet and an air outlet arranged on the sealing cover, wherein the air inlet is in communication with the sample chamber, while the air outlet is in communication with the light hydrocarbon capture unit.

In one embodiment, the air inlet is provided with an injection cannula extending into an interior of the first capture tube at a position close to a bottom portion thereof, for supplying the components to be detected to the bottom portion of the first capture tube, wherein a ratio of a length of the injection cannula extending into the interior of the first capture tube to a length of the first capture tube is 1:1.5 to 1:1.2.

In one embodiment, the first capture tube is in communication with the sample chamber through an auxiliary heating pipeline with controllable temperature, wherein an inner wall of the auxiliary heating pipeline is passivated to prevent adsorption.

In one embodiment, the testing device further comprises a gas delivery unit in communication with the sample chamber, for delivering remaining components to be detected in the sample chamber to the cold trap collection unit and the light hydrocarbon capture unit.

In one embodiment, the gas delivery unit is in communication with the sample chamber through a first gas pipeline, and the cold trap collection unit is in communication with the gas measuring unit through a second gas pipeline, wherein the light hydrocarbon capture unit is connected in the second gas pipeline, and a flowmeter is arranged between the light hydrocarbon capture unit and the gas measuring unit, for use with the gas delivery unit to control a flow rate of the carrier gas in the testing device.

In one embodiment, the light hydrocarbon capture unit includes a second capture tube filled with adsorption material.

In one embodiment, the testing device further includes a cooling gas delivery unit in communication with the sample chamber, for delivering cooling gas to the sample chamber.

According to a second aspect of the present invention, a testing method for measuring oil, gas and water contents in rock is proposed, which comprises:

Step A, providing said testing device;

Step B, weighing the first capture tube of the cold trap collection unit and the second capture tube of the light hydrocarbon capture unit;

Step C, controlling, by the central control unit, the heating desorption unit and the cold trap collection unit at respective first predetermined temperatures, so as to form an environment for gas measurement and oil-water capture testing;

Step D, placing the rock sample to be detected into the sample chamber;

Step E, recording a gas volume collected by the gas measuring unit at predetermined regular intervals until the gas volume no longer increases, and then after a predetermined time period, performing staged heating on the heating desorption unit;

Step F, adjusting the flow rate of the carrier gas in the testing device to a predetermined flow rate, and controlling, by the central control unit, the heating desorption unit and the cold trap collection unit at respective second predetermined temperatures, so as to form an environment for oil-water capture testing; and

Step G, reweighing the first capture tube and the second capture tube, and, after calculation and analysis, obtaining the oil, gas and water contents in the rock sample to be detected.

In one embodiment, in Step C, the predetermined temperature of the heating desorption unit is 60-110° C., and the predetermined temperature of the cold trap collection unit is −20-10° C.; and in Step F, the predetermined flow rate is 10-30 mL/min, the predetermined temperature of the heating desorption unit is 60-350° C., and the predetermined temperature of the cold trap collection unit is −20-10° C.

In one embodiment, in Step E, the heating desorption unit is maintained at a constant temperature after each heating, until the gas volume in the gas measuring unit no longer increases, and
the carrier gas is further delivered to the sample chamber through the gas delivery unit, so that the remaining components to be detected in the sample chamber are delivered to the cold trap collection unit and the light hydrocarbon capture unit.

Compared with the prior arts, the present application has the following advantages.

According to the testing device for measuring oil, gas and water contents in rock of the present invention, the components to be detected are respectively captured by the cold trap collection unit, the light hydrocarbon capture unit and the gas measuring unit connected in sequence. With the testing device, it is possible to test full-diameter core sample with a certain thickness at the drilling site, thus obtaining oil, gas and water contents in the rock at a time, while restoring lost gas volume based on rock gas analysis curve. The testing device will dispense with loss of gas, light hydrocarbon or moisture, which not only enhances the operating efficiency but also overcomes the defect of mismatch between testing data obtained by existing methods, thereby providing scientific basis for evaluating oil and gas content and mobility of rocks.

### BRIEF DESCRIPTION OF THE DRAWINGS

In the following, the present invention will be described with reference to the accompanying drawings.

FIG. 1 schematically shows a structure of a testing device for measuring oil, gas and water contents in rock according to the present invention.

FIG. 2 schematically shows a structure of a heating desorption unit in a sample chamber of the testing device as shown in FIG. 1.

FIG. 3 schematically shows a structure of a crushing tank cover in the heating desorption unit as shown in FIG. 2.

FIG. 4 schematically shows a structure of a first capture tube in the testing device as shown in FIG. 1.

FIG. 5 schematically shows scales and a sealing cover on the first capture tube as shown in FIG. 4.

FIG. 6 schematically shows a structure of a gas measuring unit in the testing device as shown in FIG. 1.

In the present application, all accompanying drawings are schematic ones, provided to illustrate the principle of the present invention merely, and are not necessarily drawn to actual scale.

### DETAILED DESCRIPTION OF EMBODIMENTS

The present invention will be described below in detail with reference to the accompanying drawings. It should be noted that the following detailed description is provided to illustrate the principle of the present invention merely, rather than intended to restrict the scope of protection of the present invention.

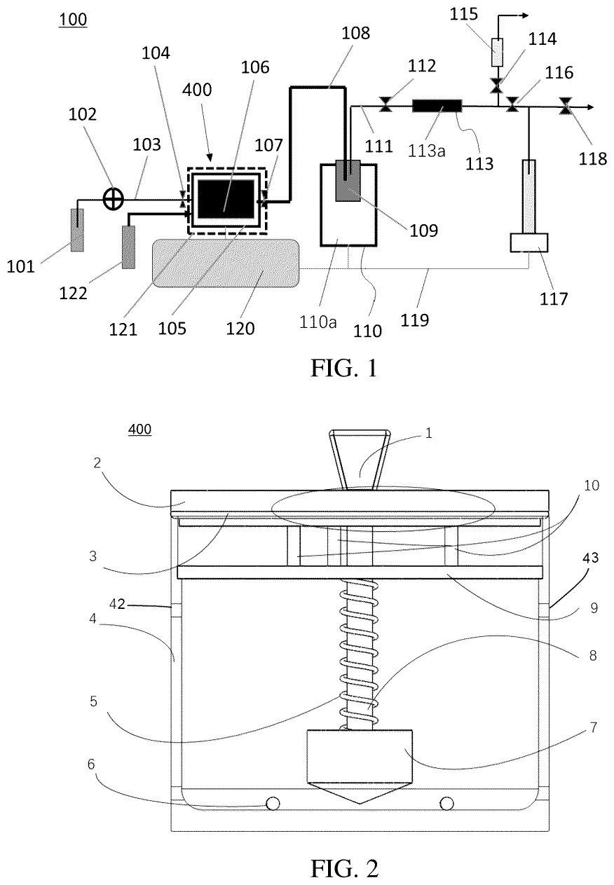

FIG. 1 schematically shows a structure of a testing device 100 for measuring oil, gas and water contents in rock according to the present invention. As shown in FIG. 1, the testing device 100 comprises a central control unit 120, and a heating desorption unit 400, a cold trap collection unit 110, a light hydrocarbon capture unit 113 and a gas measuring unit 300 connected in sequence. The heating desorption unit 400 is configured to heat a rock sample 106, in order to obtain components to be detected. The cold trap collection unit 110 is configured to trap and separate oil and water in the components to be detected, in order to obtain preliminarily separated components to be detected. The light hydrocarbon capture unit 113 is configured to trap and separate light hydrocarbon in the preliminarily separated components to be detected, in order to obtain secondarily separated components to be detected. The gas measuring unit 300 is configured to measure the gas content in the secondarily separated components to be detected. The central control unit 120 is configured to turn on and off the testing device 100.

The term “light hydrocarbon” in the present invention refers to hydrocarbon components with carbon numbers ranging from C₆ to C₁₄.

According to the present invention, as shown in FIG. 1 and FIG. 2, the heating desorption unit 400 includes a sample chamber 105 for accommodating the rock sample 106, a crushing mechanism arranged within the sample chamber 105, and a heating mechanism 121. The heating desorption unit 400 is able to crush and heat the rock sample 106 in a sealed manner, in order to achieve gas analysis and desorption analysis of oil and water contained therein, thus obtaining the components to be detected.

As shown in FIG. 2, the sample chamber 105 includes a tank body 4 and a crushing tank cover 2, which are sealed with each other by a first sealing packing, thus forming a sealed environment within the tank body 4. Preferably, an outer annular groove is provided at a bottom end of the crushing tank cover 2, so that the crushing tank cover 2 and the tank body 4 are sealed with each other by the first sealing packing filled in the outer annular groove. Further preferably, the first sealing packing may be, for example, an fluoroelastomer O-ring seal.

The crushing mechanism includes a primary crushing unit connected to the crushing tank cover 2, and a secondary crushing unit arranged inside the tank body 4 and at a bottom portion thereof. The primary crushing unit is able to impact on and crush the rock sample 106 to obtain crushed rock samples, and the secondary crushing unit is able to further attrite and grind the crushed rock samples, in order to realize secondary crushing of the rock to be crushed.

According to an embodiment of the present invention, as shown in FIG. 2, the primary crushing unit includes a push rod 8, a counterweight 7 connected to a lower end of the push rod 8, an energy storage element 5 arranged on the push rod 8, and a driving assembly 1. The counterweight 7 is arranged inside the tank body 4, and shaped as a conical cylinder with a certain weight. The primary crushing unit can move upwards and downwards, so that the counterweight 7 can hammer the rock to be crushed. An upper end of the push rod 8 passes through the crushing tank cover 2 and extends outward to connect to the driving assembly 1. Meanwhile, the push rod 8 fits with the crushing tank cover 2 in a slidable and sealed manner. The counterweight 7 can be lifted up through the driving assembly 1, so that the energy storage element 5 can store energy. Thus, when released, the counterweight 7 can impact on and crush the rock sample 106 due to its own gravity and the impact of the energy storage element 5.

The energy storage element 5 is preferably a counterweight spring arranged around the push rod 8. A bottom end of the counterweight spring is fixed to the counterweight 7, and a top end thereof abuts against a heat insulation plate 9 (see below).

The driving assembly 1 is a manual driving assembly, and may be, for example, a handle. In operation, an operator can operate the handle 1 to drive the push rod 8 to move. Of course, the driving assembly 1 may also be an electric driving assembly. For example, the electric driving assembly includes a steel wire rope connected to the handle, and a motor, a fixed pulley and a winding roller that drive the steel wire rope to move. The steel wire rope is wound around the fixed pulley. One end of the steel wire rope is connected to the winding roller which is driven by the motor, so that the motor can drive the steel wire rope to rise, enabling the energy storage element 5 and the counterweight to obtain potential energy. Subsequently, the motor stops rotating, in which case the potential energy of the counterweight and the energy storage element S is converted into the kinetic energy of the counterweight, thereby impacting on and crushing the rock sample.

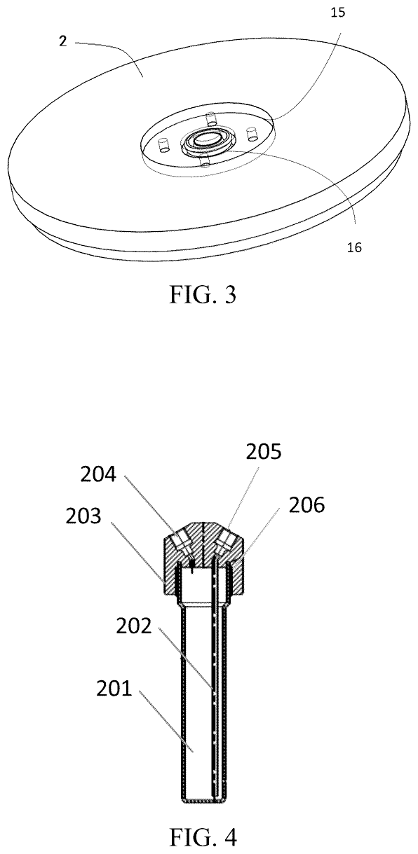

In one embodiment, as shown in FIG. 3, a dynamic seal is formed between the push rod 8 and the crushing tank cover 2 through a dynamic seal assembly, which includes a seal fixing seat 15 and a pneumatic sealing ring 16 installed thereon. The seal fixing seat 15 passes through a central area of the crushing tank cover 2, and the dynamic seal is formed between the push rod 8 and the crushing tank cover 2 through the pneumatic sealing ring 16. Specifically, the seal fixing seat 15 is embedded in a groove which is provided at a center of an upper end surface of the crushing tank cover 2, and the pneumatic sealing ring 16 is arranged in the seal fixing seat 15 to form a sliding seal with the push rod 8. Thus, the dynamic seal assembly ensures the sealing between the crushing tank cover 2 and the push rod 8.

According to an embodiment of the present invention, the secondary crushing unit includes a vibrating plate arranged at a bottom end within the tank body 4, a plurality of grinding balls 6 arranged on the vibrating plate, and an electromagnetic vibrator for driving the vibrating plate. The grinding balls 6 can move driven by the electromagnetic vibrator, in order to crush and grind the rock samples. The grinding balls 6 are preferably magnets. When a set of alternating current signals is input to the electromagnetic vibrator, a coil inside the electromagnetic vibrator will generate a corresponding alternating magnetic field, while the polarity of the magnet remains unchanged. Therefore, under the impact of the alternating magnetic field, the grinding balls 6 as permanent magnets will move violently upward, downward, leftward and rightward, thereby grinding the rock to be crushed.

Since the gas desorption of the shalein the tank body 4 occurs in a heated environment, heat exchange will be occurred between the hot gas in the tank body and an exterior thereof. As a result, part of the hot gas will condense on an inner wall of the tank body, which affects the precision of subsequent gas collection. Therefore, the heat insulation plate 9 is further fixedly installed inside the tank body 4, as shown in FIG. 2. The heat insulation plate 9 is connected to a lower end of the crushing tank cover 2 through at least one fixing rod 10 but at an interval from the crushing tank cover 2. The push rod 8 passes through a through hole formed in the middle of the heat insulation plate 9, with a second sealing packing provided therebetween to form a dynamic seal. A stepped groove is provided within the tank body 4. The heat insulation plate 9 is seated on the stepped groove and sealingly fit with the inner wall of the tank body 4 through a third sealing packing. In one embodiment, the second sealing packing and the third sealing packing may be, for example, soft graphite packing.

Preferably, the bottom end of the crushing tank cover 2 is provided with three circular grooves, each of which is fixedly arranged therein with a corresponding fixing rod 10 for fixing the heat insulation plate 9. The heat insulation plate 9 can achieve both sealing and heat insulation. In addition to the sealing function of the tank body 4 per se, the heat insulation plate 9 further seals the tank body, thus greatly improving the measurement of the desorption products of the rock.

In one embodiment, the tank body 4 is cylindrical in shape, so that the heating mechanism 121 can be arranged around the tank body 4. Preferably, the heating mechanism 121 is wound around the tank body 4, and may be, for example, a ceramic heating plate. Preferably, the heating assembly may include three ceramic heating plates, which are wound around a side portion, arranged at the bottom portion and a top portion of the tank body, respectively, for overall heating and accurate temperature control.

According to the present invention, as shown in FIG. 2, an air inlet hole 42 and an air outlet hole 43 are formed on and pass through a side wall of the tank body 4. Specifically, the air inlet hole 42 and the air outlet hole 43 are arranged on the side portion of the tank body 4 opposite each other. An imaginary line connecting the air inlet hole 42 and the air outlet hole 43 passes through an axial center of the tank body. In an embodiment as shown in FIG. 2, two air inlet holes 42 spaced apart from each other are provided along a height direction of the tank body 4, wherein the upper air inlet hole 42 is located below and close to the heat insulation plate 9, and the lower air inlet hole 42 is close to the bottom portion of the tube body 4. Meanwhile, two air outlet holes 43 spaced apart from each other are provided along the height direction of the tank body 4 and opposite the two air inlet holes 42.

The air inlet hole 42 is in communication with a purging system configured to purge and deliver oil, gas and water desorbed from the rock in the tank body 4. The air outlet hole 43 is in communication with the cold trap collection unit, the light hydrocarbon capture unit and the gas measuring unit, thereby collecting the oil, gas and water desorbed from the rock. Thus, the oil, gas and water can be purged from the sample chamber 105 thoroughly, and then delivered to the cold trap collection unit, the light hydrocarbon capture unit and the gas measuring unit, which is beneficial to improving the accuracy of the test results. The sample chamber 105 can be directly filled with full-diameter drilling rock sample with a certain thickness at the drilling site. At a temperature under experimental conditions, the gas does not leak, and the carrier gas can enter and exit without dead volume.

In one embodiment, an anti-blocking filter mesh (not shown) is provided at each of the air inlet hole 42 and the air outlet hole 43 for filtering dust. An air inlet pipe is connected to the air inlet hole 42 for supplying nitrogen, and an air outlet pipe is connected to the air outlet hole 43 for discharging oil-gas-water mixed gas. A one-way valve is provided on each of the air inlet pipe and the air outlet pipe, so that the gas will not enter the air inlet pipe after being desorped from the rock, or flow back into the tank body 4 after entering the air outlet pipe.

Preferably, the grinding ball 6 has a diameter larger than inner diameters of the air inlet hole 42 and the air outlet hole 43. Also, the air inlet hole 42 and the air outlet hole 43 are arranged higher than the grinding balls, so that the grinding balls 6 will not block the air inlet hole 42 and the air outlet hole 43.

The operation of the heating desorption unit 400 is illustrated as follows.

Firstly, the tank body 4 is filled with the rock to be crushed, and then sealed. Specifically, the crushing tank cover 2 is opened and the rock to be crushed is placed into the tank body 4. Then, the crushing tank cover 2 is placed on the tank body 4, thereby realizing the primary seal therebetween. Subsequently, the heat insulation plate 9 is encapsulated inside the tank body 4, thereby realizing the secondary seal on the tank body 4. In this manner, a two-stage seal is formed for the tank body 4. Accordingly, the oil, gas and water products desorbed from the rock can be effectively prevented from being discharged from the tank body 4 when the rock inside the tank body 4 is heated, ensuring the accuracy of the testing results.

Then, the rock to be crushed is hammered by the crushing mechanism, in order to crush the rock. Specifically, the motor drives the steel wire rope to rise, enabling the counterweight spring and the counterweight to obtain potential energy. Then the motor stops rotating, so that the potential energy of the counterweight and the counterweight spring is converted into the kinetic energy of the counterweight, thereby impacting on and crushing the rock. Alternately, the handle 1 is operated to drive the push rod 8 to move upward and downward, so that the counterweight impacts on and crushes the rock.

As the crushing mechanism hammers the rock to be crushed, the grinding balls further crush the rock in cooperation with the counterweight 7. When the counterweight 7 hammers the rock to be crushed, an extruding force will be generated between the rock to be crushed and the grinding balls. Accordingly, when the counterweight 7 hammers the rock to be crushed, small pieces of crushed rock scatter while large pieces of crushed rock move towards the center under the impact of the vibrating plate. At the same time, the grinding balls continuously attrite the crushed rock.

Then, the electromagnetic vibrator drives the grinding balls to grind the crushed core, in order to realize the secondary crushing of the core. The crushed rock is ground to 60-100 mesh, in order to realize integrated crushing.

Then, the air outlet hole 43 is opened, the tank body is heated by the ceramic heating plate, and the gas desorbed from the rock is collected by the gas measuring unit. During this procedure, the desorption gas generated inside the tank body 4 will enter the gas measuring unit, which collects and measures the rock desorption gas.

Afterwards, the air inlet hole 42 and the air outlet hole 43 are opened, in order to supply inert gas to the interior of the tank body through the air inlet hole 42. The rock desorption products are purged to the cold trap collection unit through the inert gas for oil and water collection. The inert gas in the present invention may be nitrogen, which does not blend with oil and water.

Since the rock to be crushed scatters when hammered by the counterweight 7, the heat insulation plate 9 according to the present invention can block the scattered rock and prevent it from damaging the crushing tank cover 2.

According to the present invention, as shown in FIG. 1, the cold trap collection unit 110 includes a cold trap capture mechanism 109 and a low-temperature cold trap 110a, wherein the cold trap capture mechanism 109, which is configured to capture the oil and water in the components to be detected, is arranged inside the low-temperature cold trap 110a for providing a low-temperature environment for the cold trap capture mechanism 109.

The low-temperature cold trap 110a is not limited in the present invention, as long as it can provide a low-temperature environment for the cold trap capture mechanism 109. Preferably, the low-temperature cold trap 110a may be an electrically refrigerated cold trap.

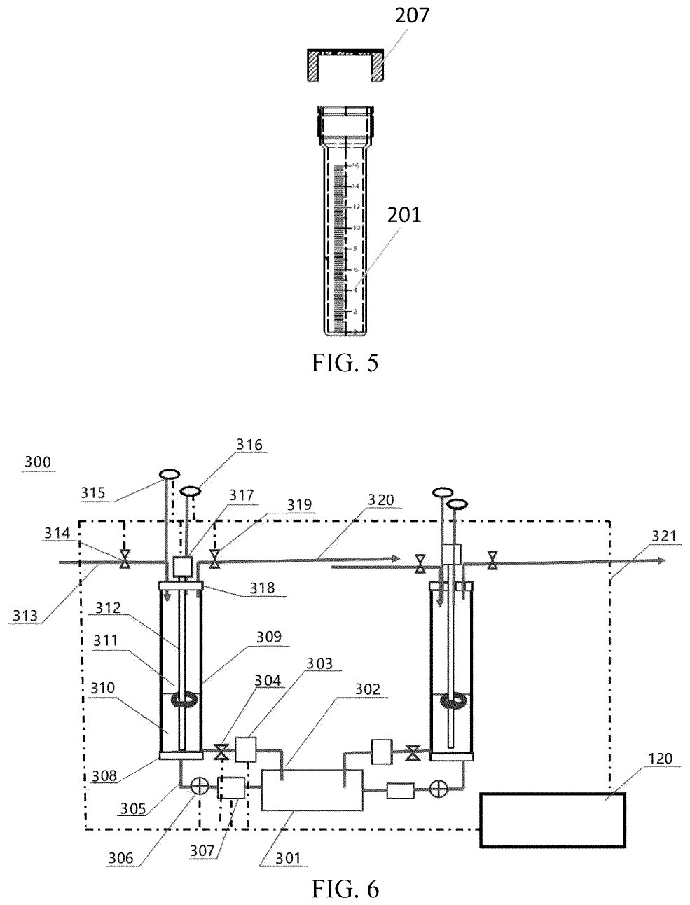

Preferably, as shown in FIG. 4 and FIG. 5, the cold trap capture mechanism 109 includes a first capture tube 201, a sealing cover 203 for sealing the first capture tube 201, an air inlet 205 and an air outlet 204 arranged in the sealing cover 203, and an injection cannula 202 connected to the air inlet 205 and extending into an interior of the first capture tube 201. A sealing ring 206 is arranged between the sealing cover 203 and the first capture tube 201, thus sealing the first capture tube 201. The air inlet 205 is in communication with the sample chamber 105, and the air outlet 204 is in communication with the light hydrocarbon capture unit 113.

According to the present invention, the injection cannula 202 extends to the interior of the first capture tube 201 and terminates at a position close to a bottom portion thereof, for supplying the components to be detected to the bottom portion of the first capture tube 201. A ratio of a length of the injection cannula 202 extending into the interior of the first capture tube 201 to a length of the first capture tube 201 is 1:1.5 to 1:1.2.

In the present invention, it is possible to increase the retention time of the components to be detected in the first capture tube 201 by adjusting the ratio of the length of the injection cannula 202 extending into the interior of the first capture tube 201 to the length of the first capture tube 201. In the same way, it is also possible to prevent the components to be detected entering the first capture tube 201 from affecting those that enters earlier, and prevent the gas flow from carrying the condensed water and oil away from the first capture tube 201, in order to avoid detection error.

According to the present invention, the light hydrocarbon capture unit 113 includes a second capture tube 113a filled with adsorption material. The second capture tube 113a filled with adsorption material is not limited in the present invention. At room temperature, only inorganic gas and gaseous hydrocarbon are allowed to pass through the second capture tube 113a, while light hydrocarbons with a carbon number above C₅ are captured therein. Under high-temperature conditions, the light hydrocarbons captured in the capture tube can be desorbed. The capture tube can be activated for reuse.

According to the present invention, the testing device 100 further comprises a gas delivery unit 101 connected to the heating desorption unit 400, for delivering the remaining components to be detected in the testing device 100 to the cold trap collection unit 110 and the light hydrocarbon capture unit 113. It should be noted that the gas delivery unit 101 herein refers to the purging system in communication with the air inlet hole 42 as mentioned above. For example, the gas delivery unit 101 may be in communication with the sample chamber 105 through a first gas pipeline 103.

The gas in the gas delivery unit 101 may be nitrogen, helium or the like. The gas used herein is not limited in the present invention, as long as it does not interfere with the detection of the components to be detected.

During the detection, a certain amount of the components to be detected will remain in the testing device 100. The gas delivered to the testing device through the gas delivery unit 101 can serve as the carrier gas for purging the thermal desorption products into the cold trap collection unit 110, the light hydrocarbon capture unit 113, etc., thereby further improving the detection accuracy of the testing device 100.

According to the present invention, the testing device 100 also comprises a flowmeter 115 arranged between the light hydrocarbon capture unit 113 and the gas measuring unit 300, and configured to work in cooperation with the gas delivery unit 101 for controlling a flow rate of the carrier gas in the testing device 100. As shown in FIG. 1, the cold trap collection unit 110 and the gas measuring unit 300 are in communication with each other through a second gas pipeline 111, in which the light hydrocarbon capture unit 113 is arranged. The flowmeter 115 is arranged between the light hydrocarbon capture unit 113 and the gas measuring unit 300 through a connecting pipe. Preferably, the flowmeter 115 is a float flowmeter.

According to the present invention, the testing device 100 further includes a cooling gas delivery unit 122 in communication with the sample chamber 105 of the heating desorption unit 400, for delivering cooling gas to the heating desorption unit 400. In this manner, the temperature in the sample chamber 105 can be reduced in a short time, so as to facilitate the detection of a next sample and thus improve the efficiency of detection.

According to the present invention, the heating desorption unit 400 is connected to the cold trap collection unit 110 via an auxiliary heating pipeline 108. Specifically, as shown in FIG. 1, the first capture tube 201 is in communication with the sample chamber 105 through the auxiliary heating pipeline 108. Preferably, an inner wall of the auxiliary heating pipeline 108 is passivated to prevent adsorption. In this manner, there will be no residue of the components to be detected in the pipeline, thus improving the accuracy of the detection.

According to the present invention, the central control unit 120 is in signal communication with each of the heating desorption unit 400, the cold trap collection unit 110 and the gas measuring unit 300 through a central control line 119, for controlling respective signals thereof.

As shown in FIG. 6, the gas measuring unit 300 includes a liquid storage tank 301, at least one measuring tube 309 in communication with the liquid storage tank 301, a magnetostrictive assembly arranged inside the measuring tube 309, and a magneto detection assembly. The magnetostrictive assembly is configured to generate a magnetostrictive effect and twist when the gas is introduced into the measuring tube 309 and a liquid level in the measuring tube 309 changes. The magneto detection assembly is configured to generate an initial pulse, and a corresponding return pulse according to the twist of the magnetostrictive assembly, in order to calculate a liquid displacement and further the gas volume.

The magnetostrictive assembly includes a detecting rod 312 vertically arranged inside the measuring tube 309, a magnetostrictive wire arranged inside the detecting rod 312, and a movable magnetic floating ring 311. When the initial pulse generated by the magneto detection assembly moves along the magnetostrictive wire, a rotating magnetic field is generated. The movable magnetic floating ring 311 is sleeved outside the detecting rod 312 and configured to move according to changes in the liquid level, for generating the magnetostrictive effect when encountering the rotating magnetic field.

According to the present invention, at least one measuring tube 309 is provided, each of which is in communication with the liquid storage tank 301, and configured to supply liquid 310 from the liquid storage tank 301 or return the liquid to the liquid storage tank 301. As shown in FIG. 6, a measuring tube base 308 is arranged at a bottom portion of the measuring tube 309, and a sealing top cover 318 is arranged at a top portion thereof, for effectively sealing the measuring tube 309, in order to ensure that the gas therein will not escape.

The liquid storage tank 301 contains liquid. When the measuring tube 309 performs drainage gas recovery, the liquid in the measuring tube 309 returns to the liquid storage tank 301. Before the measuring tube 309 performs a next operation of drainage gas recovery, liquid can be delivered to the measuring tube 309 from the liquid storage tank 301, in order to facilitate the next operation of drainage gas recovery.

Specifically, a liquid inlet pipe 302 and a liquid return pipe 305 are arranged between and in communication with the liquid storage tank 301 and the measuring tube 309. A liquid inlet pump 303 and a liquid inlet valve 304 are arranged on the liquid inlet pipe 302, and a proportional one-way speed-regulating valve 306 and a liquid return pump 307 are arranged on the liquid return pipe 305. Each of controlled ends of the liquid inlet pump 303, the liquid inlet valve 304, the proportional one-way speed-regulating valve 306 and the liquid return pump 307 is connected to an output end of the central control unit 120.

When the liquid needs to be injected, the central control unit 120 turns on the liquid inlet valve 304, activiates the liquid inlet pump 303, and turns off the proportional one-way speed-regulating valve 306. Then, the liquid inlet pump 303 drives the liquid in the liquid storage tank 301 into the measuring tube 309. When the measuring tube 309 performs the operation of drainage gas recovery, the central control unit 120 turns off the liquid inlet valve 304 but turns on the proportional one-way speed-regulating valve 306, so that the liquid in the measuring tube 309 can flow back to the liquid storage tank 301.

An air inlet assembly is arranged on the measuring tube 309 and configured to introduce gas into the measuring tube 309. A controlled end of the air inlet assembly is connected to the output end of the central control unit 120. The air inlet assembly includes an air inlet pipeline 313 and an air inlet valve 314.

Since the gas generated during the shale desorption contains moisture, introducing the gas containing moisture into the measuring tube 309 for gas measurement will affect the accuracy thereof. However, in the present invention, one end of the air inlet pipeline 313 is in communication with the top portion of the measuring tube 309, and the other end thereof is in communication with an oil-water separation device. The heating desorption unit 400 can heat the shale to desorb the gas therein. Then the desorbed gas enters the measuring tube 309 for measurement. Since in the present invention the oil-water separation device performs oil-water separation on the gas desorbed from the shale before the gas is collected, the desorbed moisture will not affect the gas collection, thus ensuring the accuracy of gas detection. It should be noted that the oil-water separation device herein includes the cold trap collection unit 110 and the light hydrocarbon capture unit 113 for oil-water separation, which can ensure that the gas entering the measuring tube 309 does not contain moisture.

The air inlet valve 314 is arranged on the air inlet pipeline 313, for turning on and off of the air inlet pipeline 313. A controlled end of the air inlet valve 314 is connected to the output end of the central control unit 120. An air outlet assembly is arranged on the measuring tube 309 and configured to discharge the gas therein. A controlled end of the air outlet assembly is connected to the output end of the central control unit 120. The air outlet assembly includes an air outlet pipe 320 and an air outlet valve 319. One end of the air outlet pipe 320 is in communication with the top portion of the measuring tube 309, and the other end thereof is in communication with the outside atmosphere. The air outlet valve 319 is arranged on the air outlet pipe 320, for turning on and off the air outlet pipe 320. A controlled end of the air outlet valve 319 is connected to the output end of the central control unit 120.

The magnetostrictive assembly is arranged inside the measuring tube 309, for generating the magnetostrictive effect and twisting when the air inlet assembly introduces gas into the measuring tube 309 and the liquid level in the measuring tube 309 changes. The magnetostrictive assembly comprises the detecting rod 312 vertically arranged inside the measuring tube 309, the magnetostrictive wire, and the movable magnetic floating ring 311. The detecting rod 312 is made of non-magnetic stainless steel tube, which can reliably protect the magnetostrictive wire. The magnetostrictive wire, i.e., waveguide wire, is arranged inside the detecting rod 312. When the initial pulse generated by the magneto detection assembly moves along the magnetostrictive wire, a rotating magnetic field is generated. The movable magnetic floating ring 311 is sleeved outside of the detecting rod 312 and configured to move according to the change in the liquid level, for generating the magnetostrictive effect when encountering the rotating magnetic field.

During operation, the magneto detection assembly can generate the initial pulse, and the corresponding return pulse according to the twist of the magnetostrictive assembly, whereby the liquid displacement and thus the gas volume can be calculated. An output end of the magneto detection assembly is connected to an input end of the central control unit 120, for transmitting a signal of the magneto detection assembly to the central control unit 120 which can monitor the signal more conveniently.

The magneto detection assembly in the present invention is an electronic chamber 317, in which an electronic circuit and an energy pickup mechanism are arranged. The electronic circuit is configured to generate pulse, and the energy pickup mechanism is configured to receive a twisting signal of the magneto detection assembly and convert it into the corresponding return pulse.

A pressure sensor 315 is further provided within the measuring tube 309, for detecting a pressure therein. An output end of the pressure sensor 315 is connected to the input end of the central control unit 120. A temperature sensor 316 is further provided within the measuring tube 309, for detecting a temperature therein. An output end of the temperature sensor 316 is connected to the input end of the central control unit 120.

In the present invention, each of the air inlet valve 314, the air outlet valve 319, the pressure sensor 315, the temperature sensor 316, the liquid inlet valve 304, the liquid inlet pump 303, the proportional one-way speed-regulating valve 306, and the liquid return pump 307 is connected to the central control unit 120 through a central control line 321.

The operation of the gas measuring unit 300 according to the present invention is illustrated as follows.

First, several measuring tubes 309 are connected to the liquid storage tank 301 in sequence, and the air inlet pipeline 313 in each measuring tube 309 is connected to a respective air outlet end of the heating desorption unit 400 and the oil-water separation device, in order to realize simultaneous gas measurement with the several measuring tubes 309, thereby increasing the speed thereof.

Then, the liquid inlet valve 304 of each measuring tube 309 is turned on. The central control unit 120 controls the liquid inlet pump 303 to pump the liquid in the liquid storage tank 301 into each measuring tube 309, and then turns off each liquid inlet valve 304.

Then, each air inlet valve 314 is turned on, while each air outlet valve 319 is turned off. The desorbed gas in the beating desorption unit 400 undergoes oil-water separation through the oil-water separation device, in order to remove oil and water contained therein. Then the gas enters each measuring tube 309 which collects the desorbed gas.

After that, the gas in the measuring tube 309 pushes the liquid therein downward. The liquid in the measuring tube 309 is returned to the liquid storage tank 301 via the liquid return pipe 305 below the measuring tube 309. As the liquid in the measuring tube 309 moves downward, the movable magnetic floating ring 311 also moves downward along with the liquid surface.

An initial pulse is generated by the electronic circuit in the electronic chamber. As the initial pulse is transmitted in the waveguide wire, a rotating magnetic field moving along the waveguide wire is generated simultaneously. When this rotating magnetic field encounters a permanent magnetic field in the movable magnetic floating ring 311, the magnetostrictive effect is generated, enabling the waveguide wire to twist. The twist is sensed by the energy pickup mechanism in the electronic chamber and converted into the corresponding return pulse. A time difference between the initial pulse and the return pulse is calculated through the electronic circuit of the electronic chamber (same as the principle of radar ranging), thus accurately measuring the liquid displacement. Then the electronic chamber feeds back the liquid displacement to the central control unit 120, which calculates the gas volume.

During the measurement through drainage gas recovery, the pressure sensor 315 can detect and feed back the pressure within the measuring tube 309 to the central control unit 120, and the temperature sensor 316 can detect and feed back the temperature within the measuring tube 309 to the central control unit 120. Therefore, the testing device according to the present invention can not only realize measurement through drainage gas recovery, but also detect the pressure and temperature of the desorbed gas, as well as changes in the pressure and temperature of the gas in the measuring tube 309.

After the measurement through drainage gas recovery is completed, the air outlet valve 319 is turned on to discharge the gas. Then, the central control unit 120 controls the liquid inlet pump 303 to pump the liquid in the liquid storage tank 301 into each measuring tube 309 again, in order to prepare for a next measurement.

The present invention further proposes a testing method for measuring oil, gas and water contents in rock, which is performed by means of said testing device 100. The testing method will be described in detail as follows with reference to FIGS. 1, 2 and 3.

Firstly, the testing device 100 is provided. A weight of the first capture tube 201 is ml, and a weight of the second capture tube 113a is m2. The first capture tube 201 and the second capture tube 113a after weighing are installed in their original positions in the testing device 100.

Next, an air-tightness test is performed on the testing device 100. Specifically, gas is supplied to the testing device 100 through the gas delivery unit 101, and the air-tightness is tested by regulating a pressure regulating valve 102, a first valve 104, a second valve 107, a third valve 112, a fourth valve 114, a fifth valve 116, and a sixth valve 118, in order to ensure that there is no leakage in the testing device 100 under a pressure of 0.3 MPa.

Then, when the carrier gas flow rate is adjusted to a predetermined flow rate, for example, a certain value within 10-30 mL/min, by regulating the pressure regulating valve 102 in cooperation with the gas measuring unit 300, the gas delivery unit 101 is turned off, in order to stop supplying gas to the testing device 100.

Subsequently, the heating mechanism 121 is set at a first predetermined temperature, for example, an actual temperature of drilling mud circulation, such as 60-110° C. According to the drilling data on site, the auxiliary heating pipeline 108 is set at a temperature within 260-300° C., and the low-temperature cold trap 110a in the cold trap collection unit 110 is set at a temperature within −20-10° C. Thus, the heating desorption unit 400 and the cold trap collection unit 110 are controlled at their first predetermined temperatures respectively, in order to form an environment for gas measurement and oil-water capture testing.

Then the first valve 104 and the second valve 107 are turned off, and the rock sample 106 is placed in the sample chamber 105. It should be noted that the rock sample 106 that needs to be crushed can be hammered and crushed by the crushing mechanism.

The central control unit 120 controls the testing device 100 to start through the central control line 119. In this case, the first valve 104, the fourth valve 114 and the sixth valve 118 are each in a closed state, the second valve 107, the third valve 112 and the fifth valve 116 are each in an open state, and the cold trap collection unit 110, the first capture tube 201 and the gas measuring unit 300 start to operate.

The open or closed state of the fifth valve 116 is adjusted at regular intervals, for example, ranging between 1-3 min, and the gas volume collected in the gas measuring unit 300 is recorded. When the gas measuring unit 300 needs to discharge gas, the fifth valve 116 is turned off and the sixth valve 118 is turned on. The discharged gas can be analyzed to detect the components thereof.

After operating for a period of time, for example, a period ranging between 1-3 h, the heating mechanism 121 is heated to 110° C. and maintained for 5-8 h, until the gas volume in the gas measuring unit 300 no longer increases. Then the fifth valve 116 and the sixth valve 118 are turned off to stop gas measurement. The first valve 104 and the fourth valve 114 are turned on, while the carrier gas is delivered to the testing device through the gas delivery unit 101 at the same time.

The heating mechanism 121 on the auxiliary heating pipeline 108 is heated to 300° C. and maintained for 1-3 h. Then the heating mechanism 121 is turned off to stop operating, and the gas delivery unit 101 stops delivering the carrier gas to the testing device. The first valve 104, the second valve 107, the third valve 112 and the fourth valve 114 are all turned off.

The carrier gas flow rate in the testing device 100 is adjusted to a predetermined flow rate through the central control unit 120, and the heating desorption unit 400 and the cold trap collection unit 110 are controlled at their second predetermined temperatures respectively, thus forming an environment for oil-water capture testing. For example, the predetermined flow rate of the carrier gas is controlled to be 10-30 mL/min, the predetermined temperature of the heating desorption unit 400 is controlled to be 60-350° C., and the predetermined temperature of the cold trap collection unit 110 is controlled to be −20-10° C.

Cooling gas is delivered to the testing device through the cooling gas delivery unit 122, in order to cool the sample chamber 105.

The second capture tube 113a is removed from the testing device and weighed as m3, wherein m3-m2 denotes a weight of light hydrocarbon in the rock. Alternately, the light hydrocarbon component can be quantitatively analyzed through a thermal desorption chromatograph.

The first capture tube 201 is removed from the testing device and weighed as m4, wherein m4-m1 denotes the oil and water contents in the rock.

A solvent (such as dichloroethylene) is added to the first capture tube 201. After shaking and layering, the reading of water layer volume is taken, in order to obtain the water content, as well as the oil content through subtraction method. In this case, the first capture tube 201 is sealed by a sealing cover 207, avoiding the liquid therein from spilling.

Also, the liquid containing oil in the first capture tube 201 can be extracted and quantitatively analyzed through the infrared method, in order to obtain the oil content, as well as the water content through subtraction method.

The gas content is a sum of the gas volume obtained in the gas measuring unit 300 and the lost gas volume. The lost gas volume can be obtained according to the rock gas desorption curve and the industry standard CN SY/T 6940-2020 “Measurement method of shale gas content”.

The total oil content in the rock is a sum of the light hydrocarbon content in the second capture tube 113a and the oil content in the cold trap capture mechanism 109.

After the sample chamber 105 is cooled, the rock sample 106 is taken out, in order to perform the testing on the next sample.

After the rock sample 106 to be detected is heated in the sample chamber 105 by the heating mechanism 121, the components to be detected in the rock sample are released into the testing device 100, and flow through the cold trap collection unit 110, the light hydrocarbon capture unit 113 and the gas measuring unit 300 in sequence. The water and oil in the components to be detected are collected by the cold trap collection unit 110, the light hydrocarbon in the components to be detected is captured by the light hydrocarbon capture unit 113, and the gas in the components to be detected are measured by the gas measuring unit 300. The testing device 100 according to the present invention is able to test full-diameter core sample with a certain thickness at the drilling site, thus obtaining oil, gas and water contents in the rock at a time, while restoring lost gas volume based on rock gas analysis curve. The testing device 100 will dispense with loss of components, which not only enhances the operating efficiency but also overcomes the defect of mismatch between testing data obtained by existing methods, thereby providing scientific basis for evaluating oil and gas content and mobility of rocks.

It should be understood that in the present invention, the terms “first” and “second” are used for illustrative purposes only, and are not intended to indicate or imply relative importance or implicitly specify the number of technical features indicated. Thus, the technical features defined with the terms “first” or “second” may explicitly or implicitly include one or more such technical features. In the description of the present invention, “a plurality of” means two or more, unless otherwise specified.

The phrases “an embodiment”, “some embodiments”, “example”, “specific example” or “some examples” as mentioned in the description mean that the particular features, structures, materials or characteristics described in conjunction with the embodiment or example are included in at least one embodiment or example of the present invention. Thus, the above illustrative phrases described throughout the description do not necessarily refer to the same embodiment or example. Moreover, the particular features, structures, materials, or characteristics described herein may be combined in any one or more of the embodiments or examples in a suitable manner.

Finally, it should be noted that the foregoing description is merely illustrative of preferred embodiments of the present invention, and is not intended to restrict the present invention. Although the present invention is described in detail with reference to the above embodiments, it is still possible for one skilled in the art to modify the technical solutions defined in the above embodiments or to replace some of the technical features with equivalent ones. Any modifications, equivalent substitutions, improvements, and the like falling within the spirit and principles of the present invention are intended to be included within the scope of protection of the present invention.

1. A testing device for measuring oil, gas and water contents in rock, comprising:
a heating desorption unit (400), comprising a sample chamber (105) for placing a rock sample (106) to be detected, a crushing mechanism arranged within the sample chamber (105), and a heating mechanism (121), the heating desorption unit (400) being configured to crush and heat the rock sample (106) in a sealed manner, in order to achieve gas analysis and desorption analysis of oil and water contained therein, thus obtaining components to be detected;
a cold trap collection unit (110) in communication with the sample chamber (105), for capturing and separating oil and water from the components to be detected, in order to obtain preliminarily separated components to be detected;
a light hydrocarbon capture unit (113) in communication with the cold trap collection unit (110), for capturing and separating light hydrocarbon from the preliminarily separated components to be detected, in order to obtain secondarily separated components to be detected;
a gas measuring unit (300) in communication with the light hydrocarbon capture unit (113), for measuring and calculating a gas volume; and
a central control unit (120), for controlling the testing device.

2. The testing device for measuring oil, gas and water contents in rock according to claim 1, characterized in that the gas measuring unit (300) includes a liquid storage tank (301), at least one measuring tube (309) in communication with the liquid storage tank (301), a magnetostrictive assembly arranged inside the measuring tube (309), and a magneto detection assembly,
wherein the magnetostrictive assembly is configured to generate magnetostrictive effect and twist when the gas is introduced into the measuring tube (309) and a liquid level in the measuring tube (309) changes, and the magneto detection assembly is configured to generate an initial pulse, and a corresponding return pulse according to a twist of the magnetostrictive assembly, whereby a liquid displacement and further the gas volume are calculated.

3. The testing device for measuring oil, gas and water contents in rock according to claim 2, characterized in that the magnetostrictive assembly comprises:
a detecting rod (312) vertically arranged inside the measuring tube (309);
a magnetostrictive wire arranged inside the detecting rod (312), wherein a rotating magnetic field is generated when the initial pulse generated by the magneto detection assembly moves along the magnetostrictive wire; and
a movable magnetic floating ring (311), which is sleeved outside the detecting rod (312) and configured to move according to changes in the liquid level, for generating the magnetostrictive effect when encountering the rotating magnetic field.

4. The testing device for measuring oil, gas and water contents in rock according to claim 2, characterized in that a pressure sensor (315) and a temperature sensor (316) are further arranged within the measuring tube (309).

5. The testing device for measuring oil, gas and water contents in rock according to claim 1, characterized in that the sample chamber (105) includes a tank body (4) and a crushing tank cover (2) connected to each other in a sealed manner through a first sealing packing.

6. The testing device for measuring oil, gas and water contents in rock according to claim 5, characterized in that the crushing mechanism includes a primary crushing unit connected to the crushing tank cover (2) and a secondary crushing unit arranged inside the tank body (4) and at a bottom portion thereof,
wherein the primary crushing unit is configured to impact on and crush the rock sample (106), in order to obtain crushed rock samples, and the secondary crushing unit is configured to attrite and grind the crushed rock samples.

7. The testing device for measuring oil, gas and water contents in rock according to claim 6, characterized in that the primary crushing unit includes a push rod (8), a counterweight (7) connected to a lower end of the push rod (8), an energy storage element (5) arranged on the push rod (8), and a driving assembly (1), an upper end of the push rod (8) passing through the crushing tank cover (2) and extending outward to connect to the driving assembly (1),
wherein the counterweight (7) is lifted up through the driving assembly (1), enabling the energy storage element (5) to store energy, and then released to impact on and crush the rock sample (106).

8. The testing device for measuring oil, gas and water contents in rock according to claim 7, characterized in that a dynamic seal is formed between the push rod (8) and the crushing tank cover (2) through a dynamic seal assembly,
wherein the dynamic seal assembly includes a seal fixing seat (15) and a pneumatic sealing ring (16) installed thereon, the seal fixing seat (15) passing through a central area of the crushing tank cover (2), so that the dynamic seal is formed between the push rod (8) and the crushing tank cover (2) through the pneumatic sealing ring (16).

9. The testing device for measuring oil, gas and water contents in rock according to claim 6, characterized in that the secondary crushing unit includes a vibrating plate, grinding balls (6) arranged on the vibrating plate, and an electromagnetic vibrator for driving the vibrating plate, wherein the vibrating plate is configured to work in cooperation with the grinding balls (6) to attrite and grind the crushed rock samples.

10. The testing device for measuring oil, gas and water contents in rock according to claim 7, characterized in that a heat insulation plate (9) is further fixed inside the tank body (4) and connected to a lower end of the crushing tank cover (2) through a fixing rod (10), wherein the push rod (8) passes through the heat insulation plate (9), with a dynamic seal formed therebetween through a second sealing packing.

11. The testing device for measuring oil, gas and water contents in rock according to claim 1, characterized in that the cold trap collection unit (110) comprises:
a cold trap capture mechanism (109), for capturing the oil and water in the components to be detected; and
a low-temperature cold trap (110a), for providing a low-temperature environment for the cold trap capture mechanism (109), the cold trap capture mechanism (109) being arranged within the low-temperature cold trap (110a).

12. The testing device for measuring oil, gas and water contents in rock according to claim 1, characterized in that the cold trap capture mechanism (109) includes a first capture tube (201), a sealing cover (203) for sealing the first capture tube (201), and an air inlet (205) and an air outlet (204) arranged on the sealing cover (203), wherein the air inlet (205) is in communication with the sample chamber (105), while the air outlet (204) is in communication with the light hydrocarbon capture unit (113).

13. The testing device for measuring oil, gas and water contents in rock according to claim 12, characterized in that the air inlet (205) is provided with an injection cannula (202) extending into an interior of the first capture tube (201) at a position close to a bottom portion thereof, for supplying the components to be detected to the bottom portion of the first capture tube (201),
wherein a ratio of a length of the injection cannula (202) extending into the interior of the first capture tube (201) to a length of the first capture tube (201) is 1:1.5 to 1:1.2.

14. The testing device for measuring oil, gas and water contents in rock according to claim 1, characterized in that the first capture tube (201) is in communication with the sample chamber (105) through an auxiliary heating pipeline (108) with controllable temperature, wherein an inner wall of the auxiliary heating pipeline (108) is passivated to prevent adsorption.

15. The testing device for measuring oil, gas and water contents in rock according to claim 1, characterized in that the testing device further comprises a gas delivery unit (101) in communication with the sample chamber (105), for delivering remaining components to be detected in the sample chamber (105) to the cold trap collection unit (110) and the light hydrocarbon capture unit (113).

16. The testing device for measuring oil, gas and water contents in rock according to claim 15, characterized in that the gas delivery unit (101) is in communication with the sample chamber (105) through a first gas pipeline (103), and the cold trap collection unit (110) is in communication with the gas measuring unit (300) through a second gas pipeline (111), wherein the light hydrocarbon capture unit (113) is connected in the second gas pipeline (111), and a flowmeter (115) is arranged between the light hydrocarbon capture unit (113) and the gas measuring unit, for use with the gas delivery unit (101) to control a flow rate of the carrier gas in the testing device.

17. The testing device for measuring oil, gas and water contents in rock according to claim 1, characterized in that the light hydrocarbon capture unit (113) includes a second capture tube (113a) filled with adsorption material.

18. The testing device for measuring oil, gas and water contents in rock according to claim 1, characterized in that the testing device further includes a cooling gas delivery unit in communication with the sample chamber (105), for delivering cooling gas to the sample chamber (105).

19. A testing method for measuring oil, gas and water contents in rock, which comprises:
Step A, providing the testing device according to claim 1;
Step B, weighing the first capture tube (201) of the cold trap collection unit (110) and the second capture tube (113a) of the light hydrocarbon capture unit (113);
Step C, controlling, by the central control unit (120), the heating desorption unit (400) and the cold trap collection unit (110) at respective first predetermined temperatures, so as to form an environment for gas measurement and oil-water capture testing;
Step D, placing the rock sample to be detected into the sample chamber (105);
Step E, recording a gas volume collected by the gas measuring unit (300) at predetermined regular intervals until the gas volume no longer increases, and then after a predetermined time period, performing staged heating on the heating desorption unit;
Step F, adjusting the flow rate of the carrier gas in the testing device to a predetermined flow rate, and controlling, by the central control unit (120), the heating desorption unit and the cold trap collection unit at respective second predetermined temperatures, so as to form an environment for oil-water capture testing; and
Step G, reweighing the first capture tube (201) and the second capture tube (113a), and, after calculation and analysis, obtaining the oil, gas and water contents in the rock sample to be detected.

20. The testing method for measuring oil, gas and water contents in rock according to claim 19, characterized in that in Step C, the predetermined temperature of the heating desorption unit is 60-110° C., and the predetermined temperature of the cold trap collection unit (110) is −20-10° C.; and
in Step F, the predetermined flow rate is 10-30 mL/min, the predetermined temperature of the heating desorption unit is 60-350° C., and the predetermined temperature of the cold trap collection unit is −20-10° C.

21. (canceled)
